(19) United States
(12) Patent Application Publication
(10) Pub. No.: US 2010/0273993 A1
(43) Pub. Date: Oct. 28, 2010

(54) METHOD FOR THE EXTRACTION OF MEMBRANE PROTEINS

(76) Inventors: Joerg Von Hagen, Pfungstadt (DE); Uwe Michelsen, Weinheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/808,015

(22) PCT Filed: Nov. 17, 2008
(86) PCT No.: PCT/EP08/09704

§ 371 Date: Jun. 14, 2010

(30) Foreign Application Priority Data
Dec. 15, 2007 (DE) 10 2007 060 599.6

(51) Int. Cl. C07K 1/14; C07H 3/00

(52) U.S. Cl. 530/412; 536/1.11

(57) ABSTRACT

The present invention relates to the use of linear, amphipathic carbohydrate polymers for the extraction of membrane proteins from biological samples, to a method for the extraction of membrane proteins, to a kit for the extraction of these proteins, and to the use thereof.

The present invention relates to the use of linear, amphipathic carbohydrate polymers for the extraction of membrane proteins from biological samples, to a method for the extraction of membrane proteins, to a kit for the extraction of these proteins, and to the use thereof.

The detection or analysis of proteins, very particularly membrane proteins, is of increasing importance in medicine. The majority of the systems investigated in pharmaceutical research comprise membrane proteins. Membrane proteins are of particular importance in a number of biological functions. Thus, many membrane proteins play a major role in the development of diseases, and consequently understanding of their function is of increasing importance in the development of medicaments. Information on the structural properties and on the function of these proteins is therefore the basis for understanding of the mechanisms.

Membrane proteins, in particular transmembrane proteins, have hydrophobic regions and are thus anchored in membranes and thus have low solubility in water. In order to facilitate in-vitro analysis of the proteins, membrane proteins are usually solubilised by addition of detergents. However, isolation of membrane proteins using detergents has the serious disadvantage that the native structure of the proteins is denatured by the influence of the detergent. Common detergents are either of an ionic nature, such as, for example, sodium dodecylsulfate (SDS), or of a nonionic nature, such as, for example, Triton-X 100. The use of SDS results in complete denaturing of all proteins and thus also of the membrane proteins, i.e. structural and functional investigations of the membrane proteins are not possible or are only possible to a very greatly restricted extent. Triton-X 100 is only capable of effectively extracting membrane proteins, in particular multiple transmembraneous proteins, in exceptional cases, and consequently the desired investigations cannot be carried out at all. A further disadvantage of detergents such as Triton-X 100 and others consists in that these reagents are not directly compatible with further analytical techniques (for example mass spectrometry).

The object was therefore to provide a method with the aid of which membrane proteins can be extracted from biological samples and investigated directly using subsequent analytical techniques.

It has now been found that water-insoluble, linear, amphipathic carbohydrate polymers, which are known for protein stabilisation and protein purification, are unexpectedly highly suitable for the extraction of membrane proteins, in particular from complex biological samples.

The present invention accordingly relates to the use of linear, amphipathic carbohydrate polymers for the extraction of membrane proteins from biological samples, since this type of polymer is insoluble in aqueous buffer systems and can be removed mechanically (for example by centrifugation, filtration, and the like) after extraction.

In a preferred embodiment, an aqueous solution comprising between 0.5 and 10% by weight of linear, amphipathic carbohydrate polymers is employed for the extraction.

In a further preferred embodiment, the membrane proteins are proteins which have one or more transmembrane passages.

In a further preferred embodiment, the biological samples are tissues, cells, cell cultures, body fluids, bacteria, fungi, viruses and/or plants.

The present invention also relates to a method for the extraction of membrane proteins from preferably native, biological samples, characterised in that a mixture at least comprising one or more linear, amphipathic carbohydrate polymers is added, optionally with mechanical action, to a preferably native, biological sample. The carbohydrate polymer is completely or virtually completely suspended in the mixture, i.e. is in undissolved form. For the method according to the invention, the carbohydrate polymer is preferably in suspension, i.e. is totally undissolved.

In a preferred embodiment, the biological sample is lysed in advance.

In a particularly preferred embodiment, the lysing of the biological samples is carried out by addition of detergents, surface-active substances and/or pore formers.

In a preferred embodiment, the mechanical action is effected by shaking or stirring.

In a further preferred embodiment, the linear, amphipathic carbohydrate polymers are uncharged.

In a preferred embodiment, the linear, amphipathic carbohydrate polymers consist of inulin or derivatives thereof.

In a further preferred embodiment, the linear, amphipathic carbohydrate polymers have a linear polyfructose backbone.

In a further preferred embodiment, the extraction is carried out at temperatures between 4 and 37° C.

In a further preferred embodiment, the concentration of the linear, amphipathic carbohydrate polymers in the extraction solution is between 0.5 and 10% by weight.

In a further preferred embodiment, the one or more linear, amphipathic carbohydrate polymers are removed by means of centrifugation, filtration, magnetic separation, sedimentation or chromatographic methods after the extraction, so that the extracted proteins remain in the resultant solution and can be subjected to further analyses without interfering influences of the extractant (=linear, amphipathic carbohydrate polymers). In a further preferred embodiment, the one or more linear, amphipathic carbohydrate polymers are employed as coating on magnetic particles in the method according to the invention.

The present invention also relates to a kit for the extraction of membrane proteins by the method according to the invention, at least comprising one or more linear, amphipathic carbohydrate polymers as solid or in liquid, and at least one lysing agent selected from the group of the detergents, surface-active substances and/or pore formers.

The present invention also relates to the use of a kit according to the invention for the extraction of membrane proteins from biological samples.

The crux of the present invention is that the method according to the invention and the kit according to the invention are suitable for the extraction of membrane proteins, in particular multipass membrane proteins, gently and as far as possible with retention of their structure. It is known to the person skilled in the art that, in particular in the case of multipass membrane proteins, function-retaining extraction from the membrane is virtually impossible since the 3D structure of the protein inevitably changes after extraction from the membrane if the transmembrane domains are removed from the hydrophobic environment of the membrane. With respect to the extraction according to the invention of multipass membrane proteins, the term “native” therefore means that, although the extracted membrane proteins are generally not extracted with retention of their function, they are, however, extracted gently and as far as possible with retention of their structure. For example, the extraction according to the invention enables mass-spectrometric and immunological measurement, in particular, of the transmembrane domains of the proteins. Using conventional methods, such as, for example, extraction with Triton-X 100, Nonidet P40 or other detergents which are used as standard, this is not possible in a comparable manner in relation to the protein yield and retention of function of the proteins to be investigated, in particular if the extract is to be further analysed directly without purification or removal of the additive.

In the case of membrane proteins which are only anchored in the membrane by means of a moiety which is irrelevant for their function or activity, such as, for example, GPI anchor proteins, “native” extraction according to the invention means that the protein can be extracted substantially with retention of its structure and activity. For example, corresponding activity measurements can be carried out in this case for detection of the protein.

Native samples are samples in which the membrane proteins to be extracted are still substantially in their native conformation, i.e. in the conformation necessary for their natural function, or samples in which the membrane proteins still exhibit activity.

The extraction of membrane proteins in accordance with the present invention can be carried out from all biological samples known to the person skilled in the art. In accordance with the invention, biological samples are all samples in which the membrane proteins are bound in a natural membrane. The biological samples are preferably tissues, such as, for example, biopsies and histological preparations, cells, cell cultures and/or cell-containing body fluids, such as, for example, blood, urine, liquor or saliva, and bacteria, plants and/or fungi. Membrane proteins from membrane-containing cell compartments or cell fragments can also be extracted in accordance with the invention. The extraction of proteins from tissues and cell cultures allows, in particular, the detection of specific proteins, for example the detection of proteins which indicate the presence of diseases. The method according to the invention is therefore also particularly advantageous for pathologically interesting tissue samples.

The method according to the invention is particularly suitable for transmembrane proteins and very particularly for multipass membrane proteins, i.e. proteins which have two or more transmembrane passages, in particular multihelical transmembrane proteins, such as, for example, heptahelical transmembrane proteins.

The class of heptahelical transmembrane proteins currently includes about 250 known proteins. The transmembrane proteins can be divided into the following sub-classes:
Class A rhodopsins, hormone proteins, (rhod)opsin, olfactory, prostanoids, nucleotide analogues, cannabinoid, platelet activating factor, gonadotropin-releasing hormones, thyrotropin-releasing hormones and secretagogues, melatonin, viral proteins, lysosphingolipid & LPA (EDG), leukotriene B4 receptors, class A orphan and others,
Class B secretins, for example calcitonin, corticotropin releasing factor, gastric inhibitory peptide, glucagon, growth hormone-releasing hormone, parathyroid hormone, PACAP, secretin, vasoactive intestinal polypeptide, diuretic hormone, EMR1, latrophilin, brain-specific angiogenesis inhibitor (BAI), Methuselah-like proteins (MTH), cadherin EGF LAG (CELSR), very large G-protein coupled receptors,
Class C metabotropic glutamate/pheromone, for example metabotropic glutamate, calcium-sensing like, putative pheromone receptors, GABA-B, orphan GPRC5, orphan GPCR6, bride of sevenless proteins (BOSS), taste receptors (T1R),
Class D fungal pheromone, for example fungal pheromone A-factor like (STE2, STE3), fungal pheromone B like (BAR, BBR, RCB, PRA), fungal pheromone M and P factor, class E cAMP receptors, frizzled/smoothened family, frizzled, smoothened and in the following non-GPCR families: Class Z archaeal/bacterial/fungal opsins.

The linear, amphipathic carbohydrate polymers employed in accordance with the invention may be linear or slightly branched. This means that, for the purposes of the present invention, lightly branched carbohydrate polymers having 1 or 2 branching points per molecule can also be taken to be linear carbohydrate polymers.

The linear, amphipathic carbohydrate polymers employed in accordance with the invention are typically completely or at least predominantly insoluble in water. They can consequently be separated off by means of simple methods, such as filtration, centrifugation, etc., after extraction.

Linear, amphipathic carbohydrate polymers which are suitable in accordance with the invention are known, for example, from WO 2005/047310.

They are preferably fructans or fructan derivatives.

Fructans are distinguished by the fact that one or more fructose molecules are bonded to a sucrose molecule.
Depending on the binding site of the fructosyl radical to the sucrose, a distinction is made between three basic types of fructan:
1-Kestoses: In inulin, the fructosyl radicals are linked to the fructosyl radical of the sucrose via β-2,1-bonds. The simplest inulin is 1-kestotriose or isokestose.
6-Kestoses: If the fructosyl radical is linked to the fructosyl radical of the sucrose via a β-2,6-bond, the term 6-kestotriose or kestose is used. Fructans of this type are sometimes known as laevans or phleins.
Neokestoses: In neokestoses or 6G-kestoses, the fructosyl radical is bonded to C6 of the glucosyl radical of the sucrose.

In the same way, the fructans or fructan derivatives employed in accordance with the invention can have various linking patterns in a molecule and thus represent a mixture of two or more basic types.

Inulin or inulin derivatives are particularly preferably employed in accordance with the invention. Further information on inulin is given, for example, in WO 2005/047310, page 3, line 4 to page 5, line 9.

Suitable inulin or inulin derivatives have a degree of polymerisation of between 3 and 500, preferably between 3 and 100, particularly preferably between 10 and 50. Particular preference is given to inulin derivatives having a degree of polymerisation of 20, 21, 22, 23, 24, 25, 26, 27, 28, 29 or 30.

The linear, amphipathic carbohydrate polymers employed in accordance with the invention preferably do not carry a charge, i.e. they have not been derivatised by charged groups.

The linear, amphipathic carbohydrate polymers employed in accordance with the invention are preferably linear carbohydrate polymers which have been mono- or polyderivatised by hydrophobic, in particular C3 to C18 alkyl chains. The carbohydrate polymers here particularly preferably consist of inulin.

Inulin derivatives which are preferred in accordance with the invention are compounds of the formula I:

G(O—CO—NH—R¹)ₐ—[F(O—CO—NH—R²)_{b}]ₙ

in which

G represents a terminal glucosyl group, in which one or more hydroxyl groups may have been derivatised, independently of one another, by a group of the formula (O—CO—NH—R¹),

R¹ is an uncharged radical, in particular a linear or branched, saturated or unsaturated hydrocarbon radical having 1 to 25 carbon atoms,

a is a number between 1 and 4,

F is a fructosyl radical, in which one or more hydroxyl groups may have been derivatised, independently of one another, by a group of the formula (O—CO—NH—R²),

R² is an uncharged radical, in particular a linear or branched, saturated or unsaturated hydrocarbon radical having 1 to 25 carbon atoms,

b is a number between 1 and 3 for fructosyl radicals within the chain and a number between 1 and 4 for terminal fructosyl radicals,

n is a number between 3 and 500, preferably between 3 and 100, particularly preferably between 10 and 50, in particular 20, 21, 22, 23, 24, 25, 26, 27, 28, 29 or 30. n is particularly preferably 24.

The average degree of derivatisation per glucosyl or fructosyl unit is between 0.02 and 3, preferably between 0.05 and 1, particularly preferably between 0.05 and 0.5.

In a preferred embodiment, the groups R¹ and/or R² are, independently of one another, alkyl, alkenyl or alkynyl groups having 1 to 25 carbon atoms, preferably 3 to 22 carbon atoms, particularly preferably 3 to 18 carbon atoms, for example n-octyl, n-decyl or n-octadecyl.

For the purposes of the invention, the term linear, amphipathic carbohydrate polymers is taken to mean a single type of linear, amphipathic carbohydrate polymers or a mixture of different linear, amphipathic carbohydrate polymers.

In a particularly preferred embodiment, the linear, amphipathic carbohydrate polymer employed in accordance with the invention is NVoy polymer, a linear, amphipathic carbohydrate polymer consisting of uncharged molecules having a molecular weight of about 5 kD which consist of linear polyfructose units which have been hydrophobically derivatised, which is commercially available from Novexin (Cambridge, GB).

The present invention relates to a method for the extraction of membrane proteins from biological samples, where at least one or more linear, amphipathic carbohydrate polymers are added to a biological sample, optionally with mechanical action, and the sample can subsequently be separated.

The at least one linear, amphipathic carbohydrate polymer added to the biological sample here is typically not in solid form, but instead in the form of an aqueous solution in which at least one linear, amphipathic carbohydrate polymer is present. The linear, amphipathic carbohydrate polymers here are typically present in the suspension in the form of a slurry. The aqueous suspension at least comprising one or more linear, amphipathic carbohydrate polymers is also known in accordance with the invention as extraction solution.

The aqueous solution used is typically water or an aqueous buffer system. It is equally possible for the aqueous solution employed in accordance with the invention to be water or an aqueous buffer system which comprises up to 20 per cent by volume of one or more water-miscible solvents.

In a preferred embodiment, an aqueous buffer system is used. This buffer system should have a pH range of between 4.5 and 9.0, preferably between 6.5 and 8.0. The pH of the solution is particularly preferably between pH 7.0 and 7.5.

Suitable buffers are all buffer systems which generate physiological conditions, i.e. do not denature proteins. Examples are PIPES, HEPES, phosphate buffers and Tris-based buffers.

The linear, amphipathic carbohydrate polymer is typically present in the aqueous solution in a concentration of between 0.5 and 10% by weight, preferably between 0.5 and 5% by weight, particularly preferably between 1 and 3% by weight.

In the simplest embodiment of the present invention, an aqueous suspension at least comprising one or more linear, amphipathic carbohydrate polymers is added to the biological sample. The said method is preferably carried out with mechanical action, for example by shaking or stirring. In this way, the extraction of the membrane proteins is accelerated and the yield of extracted proteins is improved.

In a further embodiment of the method according to the invention, the biological sample can firstly be lysed, i.e. the basic cellular structure is destroyed before use of the method according to the invention. This pretreatment can be carried out in all ways known to the person skilled in the art, for example by manual homogenisation or mechanical shaking. In particular, the lysing of the biological samples can also be carried out by addition of detergents, surface-active substances and/or pore formers known to the person skilled in the art. The prior lysing of the sample further improves the extraction result with respect to the yield of membrane proteins obtained. Thus, the membrane proteins remain in the membrane during lysing, but the membranes or membrane fragments can be separated off from the other cell constituents in a simple manner. The lysis is preferably carried out using digitonin.

The lysis can also be carried out simultaneously with the extraction, but more complex protein mixtures are then obtained.

The aqueous suspension at least comprising one or more linear, amphipathic carbohydrate polymers for use in the method according to the invention may comprise additional additives and assistants. Corresponding additives and assistants are known to the person skilled in the art and include, for example, detergents, surface-active substances, pore formers, biological or physiological buffer systems, stabilisers, mineral salts and/or inhibitors (for example protease inhibitors).

The methods according to the invention can be carried out at temperatures above 0° C., typically at between 0 and 95° C. If particularly high protein yields are to be achieved and the retention of activity or structure is secondary, the extraction can be carried out at high temperatures (above 37° C.). Extractions of this type can be utilised particularly well for Western blot analyses. The extraction according to the invention is preferably carried out at 0 to 37° C., particularly preferably at between 0 and 8° C., in particular at between 0 and 4° C. At the preferred temperatures, improved extraction in a relatively short time and the retention of the protein activity of the protein are observed. For gentle extraction, in particular of relatively sensitive membrane proteins, it is recommended that the method according to the invention be carried out at relatively low temperatures within the temperature range indicated, taking into account a longer extraction time which is necessary for this purpose.

Typical extraction times are between 30 minutes and 16 hours. If active proteins are to be extracted, the pH of the extraction solution should preferably be about pH 7.4, otherwise extraction solutions having pH values of between 2 and 10 can also be employed.

The proteins extracted in accordance with the invention can be employed directly, for example, for mass-spectrometric studies (for example Maldi, Esi and Seldi) or can also be investigated by means of all other types of protein analysis known to the person skilled in the art, for example by means of electrophoresis (for example gel electrophoresis, in particular also two-dimensional gel electrophoresis), immunochemical detection methods (for example Western blot analysis, ELISA, RIA), protein arrays (for example planar and bead-based systems), and all chromatographic separation methods, in particular biochromatographic separation methods (IEX, SEC, HIC, affinity chromatography and hydrophobic interaction chromatography), or employed in activity assays. In particular, analytical techniques for membrane protein complexes, such as blue native gel electrophoresis, plasmon resonance spectroscopy and other techniques for the analysis of protein complexes are suitable for the analysis of the proteins extracted in accordance with the invention.

The methods according to the invention are suitable for the extraction of membrane proteins from biological samples with retention of the basic cellular structure of the samples, i.e. the structure of the membrane proteins is retained as far as possible. In this way, it is also possible to isolate entire membrane protein complexes which can only be isolated with difficulty, or not at all, using conventional methods. In general, the membrane proteins obtained can be detected using suitable antibodies.

If the linear, amphipathic carbohydrate polymer is to be removed after the extraction, centrifugation, filtration, magnetic separation, sedimentation, chromatography or further physical separation methods known to the person skilled in the art are suitable for this purpose.

The linear, amphipathic carbohydrate polymer can also be employed in the method according to the invention in immobilised form, i.e. as coating of surfaces or preferably particles. In particular, magnetic particles coated with the linear, amphipathic carbohydrate polymer are preferred here. For example, these may be magnetite or maghaemite particles having particle sizes of between 5 and 100 nm. The carbohydrate polymer coating may be applied covalently or non-covalently to the particles. For example, the particles are firstly coated with a bond coat of, for example, silica or an organic polymer, to which the linear, amphipathic carbohydrate polymer is then applied.

The present invention likewise relates to a kit for the extraction of membrane proteins by the method according to the invention described above, comprising at least one linear, amphipathic carbohydrate polymer and a lysing agent. The kit typically comprises the linear, amphipathic carbohydrate polymer
in solid form,
in the form of a highly concentrated slurry of the carbohydrate polymer in water, a water-miscible organic solvent (for example ethanol) or an aqueous buffer system
or in the form of an aqueous suspension, as described above under performance of the method according to the invention.

In general, the kit additionally also comprises a suitable buffer, in which the carbohydrate polymer can be suspended in suitable amount for the extraction. Further optional constituents are, for example, protease inhibitors or wash buffers. Suitable lysing agents have already been described. Digitonin is particularly preferably employed.

The kit according to the invention enables the user to extract membrane proteins from biological samples in a simple manner.

The present invention likewise relates to the use of the kit according to the invention for the extraction of membrane proteins, in particular multiple transmembraneous proteins, from biological samples.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below, in particular the corresponding application DE 10 2007 060599.6, filed on Dec. 15, 2007, is incorporated into this application by way of reference.

### EXAMPLE

Extraction According to the Invention of Membrane Proteins from HEK 293 Cells

| | |
|---|---|
| Wash buffer | 1X PBS |
| Protease inhibitor cocktailset III | Calbiochem. Art. No.: 539134 |
| Extraction buffer I | 10 mM PIPES pH 6.8 |
| | 0.02% by weight of digitonin |
| | 300 mM sucrose |
| | 15 mM NaCl |
| | 0.5 mM EDTA |

Extraction buffer I can optionally be employed for extracting the cytosolic proteins in advance.

| | | |
|---|---|---|
| Extraction buffer II | 10 | mM PIPES pH 7.4 |
| | 300 | mM sucrose |
| | 15 | mM NaCl |
| | 0.5 | mM EDTA |

### Preparation of the Carbohydrate Polymer (NVoy Polymer (Novexin, GB))

Transfer 500 μl of the polymer (suspension in ethanol) into an Eppendorf cup
Centrifuge at 5000 g for 3 min
Discard the supernatant, wash the polymer with 1000 μl of extraction buffer II
Centrifuge at 5000 g for 3 min, discard the supernatant
Repeat twice
At the end prepare an approx. 1:1 slurry (50% of polymer/50% of buffer)
For the extraction, prepare a 2% solution (% by vol.) in extraction buffer II therefrom

### Procedure in the Case of Adherent Cells:

T₇₅ bottles containing, for example, HEK 293 cells (about 90% covered)
Pour off the medium
Wash 2× with 10 ml of PBS (4° C.), discard the wash buffer (between 0 and 37° C.)
+10 μl of protease inhibitor cocktail (optional)
add 1000 μl of 2.0% (% by vol.) solution of polymer per bottle, distribute carefully
leave to stand for 30 min at 4° C. (between 10 and 60 min and between 0° C. and 37° C.)
scrape off the cells, centrifuge at 16,000 g for 15 min at 4° C.
   Besides cellular compartments, the polymer is also centrifuged off and removed from the sample. =>the supernatant can then be added as protein fraction for further analysis (for example direct mass-spectrometric investigation, and analytical techniques of membrane protein complexes (for example blue native gel electrophoresis) and all native enzymatic and immunological analytical methods).

The method can also be carried out in the same way, for example, with suspension cells, isolated tissue cells or homogenised tissues.

1. Use of at least one linear, amphipathic carbohydrate polymer for the extraction of membrane proteins from biological samples.

2. Use according to claim 1, characterised in that an aqueous suspension comprising between 0.5 and 10% by weight of at least one linear, amphipathic carbohydrate polymer is employed for the extraction.

3. Use according to claim 1, characterised in that the biological samples are tissues, cells, cell cultures, body fluids, bacteria, fungi, viruses and/or plants.

4. Method for the extraction of membrane proteins from biological samples, characterised in that a mixture at least comprising one or more linear, amphipathic carbohydrate polymers is added to a biological sample.

5. Method according to claim 4, characterised in that the biological sample is lysed before the addition of the mixture at least comprising one or more linear, amphipathic carbohydrate polymers.

6. Method according to claim 4, characterised in that the one or more linear, amphipathic carbohydrate polymers present are uncharged.

7. Method according to claim 4, characterised in that the one or more linear, amphipathic carbohydrate polymers present consist of inulin or derivatives thereof.

8. Method according to claim 4, characterised in that the one or more linear, amphipathic carbohydrate polymers present have a linear polyfructose backbone.

9. Method according to claim 4, characterised in that the extraction is carried out at temperatures between 4 and 37° C.

10. Method according to claim 4, characterised in that the concentration of the linear, amphipathic carbohydrate polymers in the suspension or mixture is between 0.5 and 10% by weight.

11. Method according to claim 4, characterised in that the one or more linear, amphipathic carbohydrate polymers are removed by means of centrifugation, filtration, magnetic separation, sedimentation or chromatographic methods after the extraction.

12. Method according to claim 4, characterised in that the one or more linear, amphipathic carbohydrate polymers are applied to magnetic particles.

13. Kit at least comprising one or more linear, amphipathic carbohydrate polymers and at least one lysing agent selected from the group of the detergents, surface-active substances and/or pore formers.

14. Use of a kit according to claim 13 for the extraction of membrane proteins from biological samples.
